(19) United States
(12) Patent Application Publication
(10) Pub. No.: US 2025/0213113 A1
(43) Pub. Date: Jul. 3, 2025

(54) MEDICAL IMAGE PROCESSING SYSTEM, METHOD, AND COMPUTER READABLE MEDIUM THEREOF

(71) Applicants: Far Eastern Memorial Hospital, New Taipei City (TW); YUAN ZE UNIVERSITY, Taoyuan City (TW)

(72) Inventors: Elsa Lin-Chin Mai, New Taipei City (TW); Tai-Yuan Su, Taoyuan City (TW); Bing-Hong Chen, Taoyuan City (TW)

(73) Assignees: Far Eastern Memorial Hospital, New Taipei City (TW); YUAN ZE UNIVERSITY, Taoyuan City (TW)

(21) Appl. No.: 18/988,836

(22) Filed: Dec. 19, 2024

Related U.S. Application Data
(60) Provisional application No. 63/617,082, filed on Jan. 3, 2024.

(30) Foreign Application Priority Data
Jan. 3, 2024 (TW) 113100236

(51) Int. Cl. A61B 3/117; A61B 3/12; A61B 3/14; G06T 7/00; G06V 10/25; G06V 10/60; G06V 40/18

(52) U.S. Cl. CPC A61B 3/1176; A61B 3/12; A61B 3/14; G06T 7/0012; G06V 10/25; G06V 10/60; G06V 40/193; G06V 40/197; G06T 2207/20084; G06T 2207/20132; G06T 2207/30041

(57) ABSTRACT

A medical image processing system and method, and a computer readable medium for processing a medical image are provided, which can be used for detecting, classifying, and/or assisting in diagnosing cataract. A data acquisition module is used to acquire an ultra-wide field fundus image, a cropping module is used to crop the ultra-wide field fundus image into a cropped image, and a deep learning module is used to detect and determine a classification of a lens opacification type corresponding to the cropped image. Therefore, an automatic screening for cataract can be realized with an increased detection rate and a decreased false negative rate, and ophthalmologists can thus cut down diagnosis time with increased examination efficiency. Also, telemedicine can be achieved accordingly.

### CROSS REFERENCE TO RELATED APPLICATIONS

This application claims the benefit of U.S. Provisional Application No. 63/617,082, filed on Jan. 3, 2024 and Taiwan Patent Application No. 113100236, filed on Jan. 3, 2024. The content of the application is incorporated herein by reference.

### BACKGROUND OF THE INVENTION

### 1. Field of the Invention

The present disclosure is related to medical diagnosis techniques, particularly to a medical image processing system, method, and computer readable medium thereof for detecting and determining classification of a lens opacification type.

### 2. Description of the Prior Art

Cataract is a common eye disease. The lens of patients suffering from this disease may become cloudy due to chemical reactions and have opaque substances blocking eye sight of the patient, thereby resulting in visual impairment or blindness. Cataract cannot be prevented in advance and is an age-related disease that mostly occurs in the elderly. However, patients suffering from cataract can usually regain their vision after undergoing lens replacement surgery.

Artificial intelligence is one solution for assisting eye disease diagnosis. For example, artificial intelligence may be integrated with image analysis to classify level (e.g., 4 levels) of diabetic retinopathy and can help clinicians conduct a more precise diagnosis. However, a mature resolution for detecting and classifying via artificial intelligence in aspect of cataract diagnosis has not been developed yet.

Furthermore, a large amount of time is wasted in obtaining pathological information during the ophthalmology consultation process. For example, the current cataract diagnosis process involves steps of: a patient putting mydriatic agents to dilate his/her pupils, a clinician inspecting the dilated pupils of the patient using a slit lamp, the clinician identifying position and formation of opaque substance in the lens of the patient to determine type and level of cataract of the patient, and the clinician determining a corresponding treatment (e.g., requirement of surgery, means for carrying out the surgery, etc.) for the patient. The above diagnosis process is greatly depending on experience of the clinician, and it is difficult for the clinician to explain the condition to the patient in a visualized manner.

In addition, traditional fundus image lacks information content due to its shooting angle (usually about 45 degrees) and is not effective for classifying the type of cataracts. For example, a traditional fundus image of arbitrary cataract patient may, at best, present a degree of ambiguity for determining severity of cataract.

Moreover, in the case of insufficient resident medical professionals in rural areas, elderly patients with cataracts and limited mobility are more hesitant towards admission to hospital for examination due to long transportation time, thus tend to delayed treatments.

Take treating patients with posterior polar cataract (PPC) as an example, patients with this type of cataract are prone to develop posterior capsular rupture (PCR), which increases the risk and complexity of surgery and prolongs the time in operating room, wasting medical resource. Therefore, if a technique can be developed to assist clinicians in detecting and classifying cataract using existing devices and simultaneously remind the clinicians of presence of PPC before surgery, the clinicians can promptly respond or change surgical tactics, which can significantly reduce occurrence of the PCR complications during the surgery.

Therefore, there is an unmet need in the art to develop a medical image processing technique that automatically assists clinicians to detect and classify the type and level of cataract after data is acquired from existing instruments.

### SUMMARY OF THE INVENTION

In view of the foregoing, the present disclosure provides a medical image processing system having a data acquisition module, a cropping module coupled with the data acquisition module, and a deep learning module coupled with the cropping module. The data acquisition module is used to acquire an ultra-wide field fundus image. The cropping module is used to crop the ultra-wide field fundus image into a cropped image. The deep learning module is used to detect and determine classification of a lens opacification type corresponding to the cropped image.

Also provided in this disclosure is a medical image processing method including: a data acquisition module acquiring an ultra-wide field fundus image, a cropping module cropping the ultra-wide field fundus image into a cropped image, and a deep learning module detecting and determining classification of a lens opacification type corresponding to the cropped image.

Further provided in this disclosure is a computer readable medium storing computer executable instruction, where the computer executable instruction is executed to perform the medical image processing method of the present disclosure.

The present disclosure will become obvious to those of ordinary skill in the art after reading the following detailed description of the embodiments illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustrative diagram of elements of a medical image processing system in accordance with at least one embodiment of the present disclosure.

FIG. 2 is a flowchart of a medical image processing method in accordance with at least one embodiment of the present disclosure.

FIG. 3 is an illustrative diagram of an ultra-wide field fundus image in accordance with at least one embodiment of the present disclosure.

FIG. 4 is an illustrative diagram of shadow features in an ultra-wide field fundus image in accordance with at least one embodiment of the present disclosure.

FIG. 5 is a flow chart for steps of acquiring experiment data in accordance with at least one embodiment of the present disclosure.

FIG. 6 is an illustrative diagram of cropping the ultra-wide field fundus image in accordance with at least one embodiment of the present disclosure.

FIG. 7 is an illustrative diagram of information content of an ultra-wide field fundus image in accordance with at least one embodiment of the present disclosure.

FIG. 8 is an illustrative diagram of arrangement of datasets in accordance with at least one embodiment of the present disclosure.

FIG. 9 is an illustrative diagram of elements of a neural network model in accordance with at least one embodiment of the present disclosure.

FIG. 10 to FIG. 13 are illustrative diagrams regarding an experiment result in accordance with at least one embodiment of the present disclosure.

FIG. 14 to FIG. 16 are illustrative diagrams regarding another experiment result in accordance with at least one embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

As used herein, “comprising”, “including”, or “having” a specific element, unless otherwise specified, may include other elements such as components, ingredients, structures, regions, portions, devices, systems, steps, or connection relationships rather than exclude those elements.

The terms “first,” “second,” etc., used herein are simply used to describe or distinguish elements such as data, components, ingredients, or structures, rather than used to limit the scope of implementation of the present disclosure or to limit the order of the elements. In addition, unless otherwise specified, the singular forms “a” and “the” used herein also include plural forms, and the terms “or” and “and/or” used herein are interchangeable.

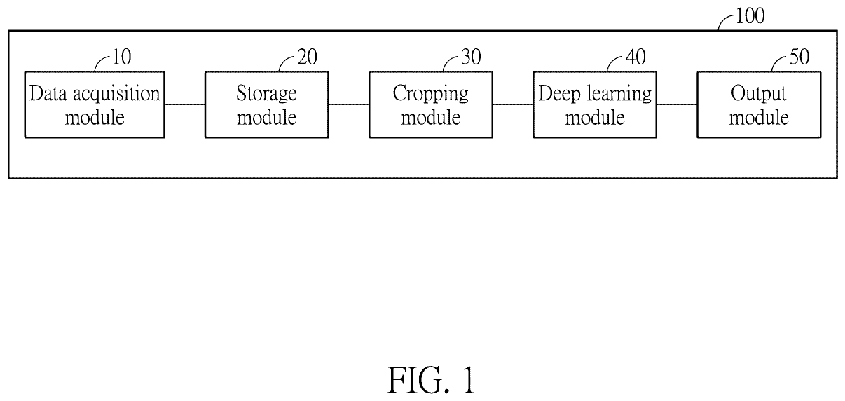

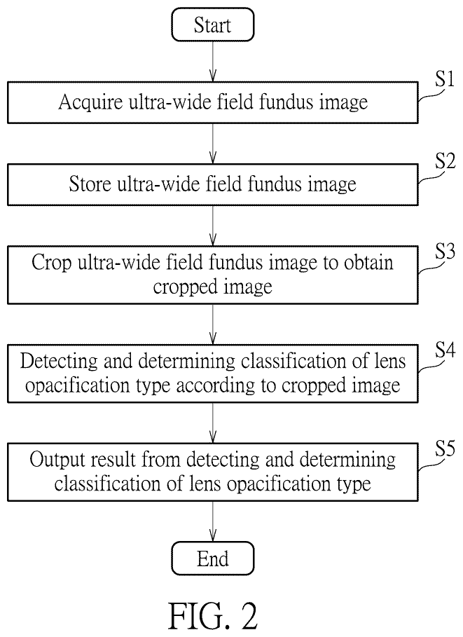

FIG. 1 describes a medical image processing system 100 according to at least one embodiment of the present disclosure. The medical image processing system 100 may include a data acquisition module 10, a storage module 20, a cropping module 30, a deep learning module 40 and an output module 50. The aforementioned elements of the medical image processing system 100 may be coupled with each other via any appropriate wired or wireless manner, and the present disclosure is not limited thereto. Further, FIG. 2 may be simultaneously referenced to understand operational relationships between the elements of the medical image processing system 100.

The data acquisition module 10 may be coupled to a fundus photography system, in particular an ultra-wide field fundus photography system, and is used to acquire an ultra-wide field fundus image of a patient (Step S1). For example, a patient may pay a visit to an eye clinic or a facility equipped with the ultra-wide field fundus photography system and request for ultra-wide field fundus image to be photographed, an operator at the eye clinic or the facility may operate the ultra-wide field fundus photography system to take an ultra-wide field fundus image for the patient, the data acquisition module 10 may acquire the ultra-wide field fundus image from the ultra-wide field fundus photography system in real-time, and the ultra-wide field fundus image may be passed on to other elements of the medical image processing system 100 for detecting and classifying a lens opacification for the patient. In at least one embodiment, the ultra-wide field fundus image may be a colored ultra-wide field fundus image.

The storage module 20 is coupled to the data acquisition module 10 and may be used to store and maintain the ultra-wide field fundus image acquired from the data acquisition module 10 (Step S2). For example, the ultra-wide field fundus image of the patient may be accessed by other elements of the medical image processing system 100 right after being stored at the storage module 20. In another example, the ultra-wide field fundus image of the patient may be stored in the storage module 20 first, a clinician performing examination (e.g., via a remote medical service) on the patient may then access the ultra-wide field fundus image through operating the medical image processing system 100 using an user interface (not shown). Further, the storage module 20 may store the ultra-wide field fundus image for other applications such as: enabling clinicians to access original image and demonstrate symptoms to the patient, preserving the ultra-wide field fundus image for a predetermined period of time in case of medical disputes, or providing the ultra-wide field fundus image as training set for improving processing efficacy of the deep learning module 40 of the medical image processing system 100. The storage module 20 may be realized as any appropriate data storage device, system, database, cloud storage, or the like, the present disclosure is not limited thereto.

The cropping module 30 is coupled to the storage module 20 and may be used to access the ultra-wide field fundus image stored in the storage module 20 and crop the ultra-wide field fundus image into a cropped image (Step S3). For example, before the ultra-wide field fundus image of the patient is used for detecting and classifying a lens opacification (cataract) for the patient, the cropping module 30 may perform center cropping of the ultra-wide field fundus image to obtain a cropped image having a region of interest of eye region in the ultra-wide field fundus image. The cropped image resulting from processing of the cropping module 30 may eliminate excessive information in the ultra-wide field fundus image and improve efficacy of the deep learning module 40. The cropped image may be a square of 1400×1400 pixels, or other sizes or shapes, the present disclosure is not limited thereto.

The deep learning module 40 is coupled to the cropping module 30 and may be used to detect and classify lens opacification for the patient using the cropped image obtained from the cropping module 30. For example, the deep learning module 40 may carry a neural network model based on transfer learning (e.g., a neural network model established using ConvNeXt-Tiny as a pre-trained neural network) to detect and determine classification of a lens opacification type by analyzing the region of interest in the cropped image. In at least one embodiment of the present disclosure, the lens opacification type corresponds to a type of cataract. In some embodiments, the deep learning module 40 detecting and determining classification of a lens opacification type may be based on analyzing the region of interest in the cropped image to correspond to one of the following: cataract with a specific type characteristic, cataract without the specific type characteristic, or non-cataract. However, the deep learning module 40 may be configured to analyze cataract based on more than one type characteristic (e.g., the deep learning module 40 may be used to distinguish more than two types of cataract of different type characteristics), and may be based on neural network module established from other pre-trained neural networks.

The output module 50 is coupled to the deep learning module 50 and may be used to generate output according to result of the deep learning module 40 detecting and determining classification of a lens opacification type (e.g., the deep learning module 40 analyze the cropped image and determine the corresponding classification of the lens opacification type) (Step S5). For example, after the deep learning module 40 has detected and determined classification of a lens opacification type corresponding to the cropped image, the output module 50 may output the lens opacification type on a human-machine interface in visualized manner for viewing by the clinician. The human machine interface may present the lens opacification type, the cropped image (ultra-wide field fundus image), and related data of the patient in a form of diagnostic report. Or, the human-machine interface may visually stack information of the lens opacification type and/or the related data of the patient on the cropped image or the original ultra-wide field fundus image for viewing by the clinician and/or projection onto screen for discussion of illness and treatment with the patient. However, output generated by the output module 50 may be realized by other appropriate means, and the present disclosure is not limited thereto.

In some alternative embodiments, the elements of the medical image processing system 100 may be realized as appropriate computing device, apparatus, application, system or the like, and the present disclosure is not limited thereto. For example, any two or more of the data acquisition module 10, the storage module 20, the cropping module 30, the deep learning module 40 and the output module 50 may be integrated instead of acting as an independent element. However, arrangement for the elements of the medical image processing system 100 may be realized through any appropriate manner and the present disclosure is not limited thereto.

In other embodiments of the present disclosure, there also exists a computer readable medium storing computer executable instruction, where the computer executable instruction is executed to perform the method of the present disclosure.

An embodiment regarding detecting and classifying cataract is described below to demonstrate working mechanisms of the data acquisition module 10, the storage module 20, the cropping module 30, the deep learning module 40 and the output module 50 of the present disclosure.

### Methodology

### Ultra-Wide Field Fundus Image

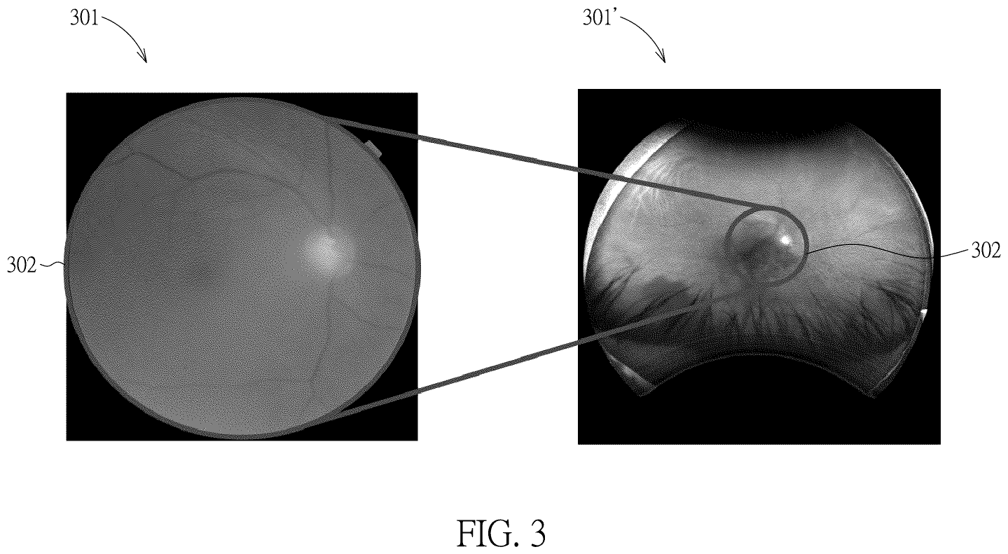

Ultra-wide field fundus photography is a technique developed from the past 10 years. FIG. 3 depicts differences between ultra-wide field fundus photography and traditional fundus photography while photographing a same patient with posterior subcapsular cataract, respectively. The left image 301 of FIG. 3 shows an area centered at center position 302 of retina of the patient photographed by a traditional fundus camera having shooting angle of 45°. The right image 301′ of FIG. 3 shows an area centered at center position 302 of retina of the patient photographed by an ultra-wide field fundus camera having a shooting angle of 200°. It can be seen that ultra-wide field fundus photography is superior to traditional fundus photography as it can not only photograph area at center position 302 of retina but also record information at outer edge of the retina. Therefore, ultra-wide field fundus photography may enable new diagnosis means for clinicians and simplify diagnosis for lens opacification.

Wide field-of-view of ultra-wide field fundus photography may enable more possibilities for diagnosis. Looking back at medical history of using fundus image as diagnosis means, cataract may result in opaque substances generated in lens, the opaque substance may block light from penetrating and shadowing the retina. However, shadow shown in images 301 and 301′ of FIG. 3 is completely different from each other. For example, traditional fundus photography is limited by a smaller field-of-view and can only present an ambiguous image 301 of cataract of the patient. On the other hand, the image 301′ obtained through ultra-wide field fundus photography may clearly present edges, type and positions of shadow on the retina. Therefore, at least one embodiment of the present disclosure utilizes shadow features (i.e., projected features on the retina caused by cataract) of image 301′ obtained through ultra-wide field fundus photography as reference for detecting and classifying cataract.

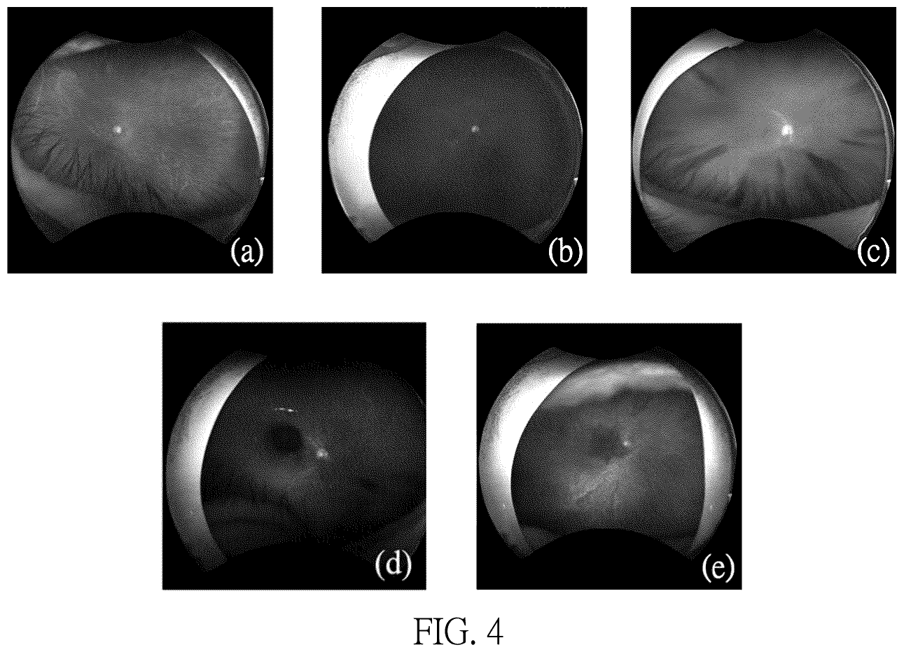

Moreover, FIG. 4 shows different shadow features (i.e., projected features on the retina caused by cataract) present under ultra-wide field fundus image caused by different type of cataract: (a) of FIG. 4 is an ultra-wide field fundus image of retina without cataract and no presence of shadows; (b) of FIG. 4 is an ultra-wide field fundus image of retina with presence of nuclear cataract, where crystal nucleus of lens becomes tawny and low in transmittance and makes the image quality to be low in brightness and can only show ambiguous blood vessel pattern of the retina; (c) of FIG. 4 is an ultra-wide field fundus image of retina with presence of cortical cataract, where feather-like opaque substance are generated at edges of the lens to cause shadow with corresponding shape to appear; (d) of FIG. 4 is an ultra-wide field fundus image of retina with presence of posterior polar cataract, where discrete, dense and rounded specks are generated at center of the lens to cover macula and cause speckled shadows to appear at center of image; and (e) of FIG. 4 an ultra-wide field fundus image of retina with presence of posterior subcapsular cataract, where opaque substance is at center of the lens to cause shadow to appear at center of the image.

The following description details embodiments of detecting and classifying posterior subcapsular cataract or posterior polar cataract for a patient. However, those with common knowledge in the art should understand that the present disclosure is also applicable for detecting and classifying cataract with other type characteristics, such as those shown in FIG. 4 or other types of cataract (e.g., diabetic cataract).

### Coaxial Lighting Operation Microscope

Coaxial lighting operation microscope is a visualization apparatus to enable visual observation during operation for eye clinicians. Coaxial lighting operation microscope utilizes coaxial lighting imaging technique to adjust imaging positions back and forth and obtain a clearer field-of-view to observe eye of a patient than with naked eye. This type of apparatus also possesses video recording functionality, and is beneficial for clinicians to record and archive surgical process completely, which in turn is convenient for acting as training material for intern clinicians or means for clarifying disputes in case of a subsequent medical dispute.

Eye of a patient will be fully dilated during operation, and red reflection caused by coaxial lighting of the coaxial lighting operation microscope irradiating on fundus may be used to observe positions of cataract characteristics for the patient, and the positions of cataract characteristics may be used to determine a corresponding type of the cataract. Moreover, clinician may diagnose nuclear cataract for the patient by determining hardness of emulsion through process of capsular being open shown in video via the recording functionality. Based on the aforementioned benefits, the video under coaxial lighting operation microscope based on operation coaxial photography is being used as ground truth for training neural network model of the deep learning module 40 for distinguishing the type of cataract.

### Experiment Subjects

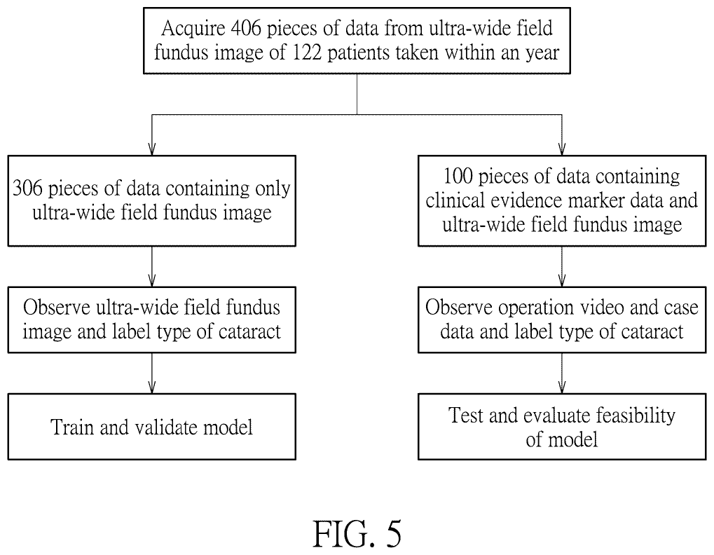

FIG. 5 is a schematic diagram illustrating means for acquiring dataset for establishing the neural network model of the deep learning module 40. The embodiment of the present disclosure acquires experiment and research data from eye clinic department of Far Eastern Medical Foundation Far Eastern Memorial Hospital. The experiment and research data is retrospectively acquired from 122 patients having ultra-wide field fundus image taken during a period of one year, which is consisted of 406 pieces of data. The dataset of 406 pieces of data may be partitioned into two groups (i.e., a first dataset and a second dataset) based on whether a piece of data is accompanied with clinical evidence marker data. Further, since posterior subcapsular cataract (PSC) is selected to be targeted type of cataract for evaluation, each piece of data in the first dataset and the second dataset may be labeled as one of the three categories: cataract with PSC characteristics, cataract without PSC characteristics, and non-cataract (control group). The description below details means for labeling each piece of data for the first dataset and the second dataset:

I. First Dataset with Data Having Only Ultra-Wide Field Fundus Image (306 Pieces)

Data in the first dataset only records ultra-wide field fundus image of the patient before operation for cataract and does not record type of the cataract. Therefore, eye clinicians may observe the ultra-wide field fundus image and label the data as one of the three categories as discussed above based on experience. In the present disclosure, the first dataset may act as training set and validation set for establishing the neural network model and is beneficial for the neural network model to learn classification logics of the eye clinicians.

### II. Second Dataset Having Clinical Evidence Marker Data (100 Pieces)

The data in this second dataset not only records ultra-wide field fundus image of the patient before operation for cataract, but also records at least one of operation coaxial photography video and medical record regarding confirmed type of cataract of the patient (collectively referred as “clinical evidence marker data”). The clinical evidence marker data may act as ground truth for clinically determining type of cataract. In the present disclosure, the ultra-wide field fundus image in data of the second dataset will not be used to train the neural network model, but will act as testing set for evaluating generalization ability of the neural network model and feasibility of using ultra-wide field fundus image for detecting and classifying type of cataract.

### Structure of Experiment

### I. Selection for Base Model for Transfer Learning and Size for Center Cropping

The neural network model of deep learning module 40 of the present disclosure is established based on base model for transfer learning. For example, a convolutional neural network model for image classification (e.g., ResNet50, InceptionV3, Xception, EfficientNetV2-S, ConvNeXt-Tiny, or the like) may be selected based on its efficacy to act as base model for transfer learning, and the selected convolutional neural network model may be used to establish neural network model of the deep learning module 40 for detecting and classifying types of cataract.

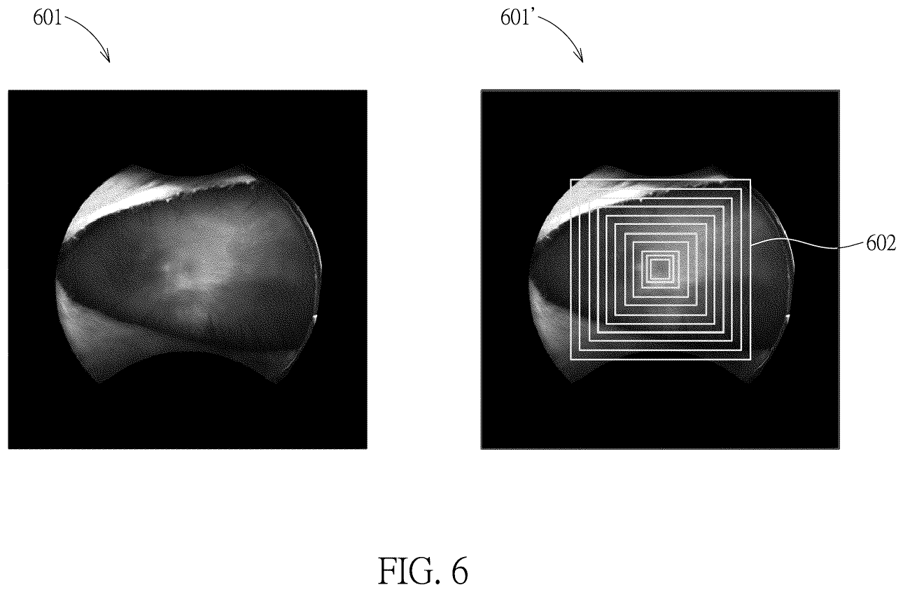

Size for center cropping is also set for cropping module 30 of the present disclosure to enable optimal processing efficacy for the deep learning module 40. Refer to FIG. 6, the image 601 on the left represents an ultra-wide field fundus image before cropping, where upper eyelid, lower eyelid and eyelash of non-fully opened eye of the patient is present in the image 601. On the other hand, the image 601′ on the right of FIG. 6 is a cropped image after center cropping processing by the cropping module 30, where unnecessary information is eliminated to avoid disturb and secure efficacy in identification for neural network model of the deep learning module 40. In here, the center cropping processing performed by the cropping module 30 may crop the ultra-wide field fundus image into a squared region (i.e., region of interest 602) that eliminates redundant information without losing necessary features. Therefore, the cropping module 30 may obtain the cropped image containing the region of interest 602 and send the cropped image to the deep learning module 40.

For example, the image 601 on the left of FIG. 6 is a three-channel colored image with original size of 4000×4000 pixels. On the other hand, the image 601′ on the right of FIG. 6 describes that region of interest 602 for cropping from the three-channel colored image may be selected from a square with side length of 300 pixels, a square with side length of 400 pixels, or a square with side length of 400+200×n≤2000 pixels, n being a positive integer.

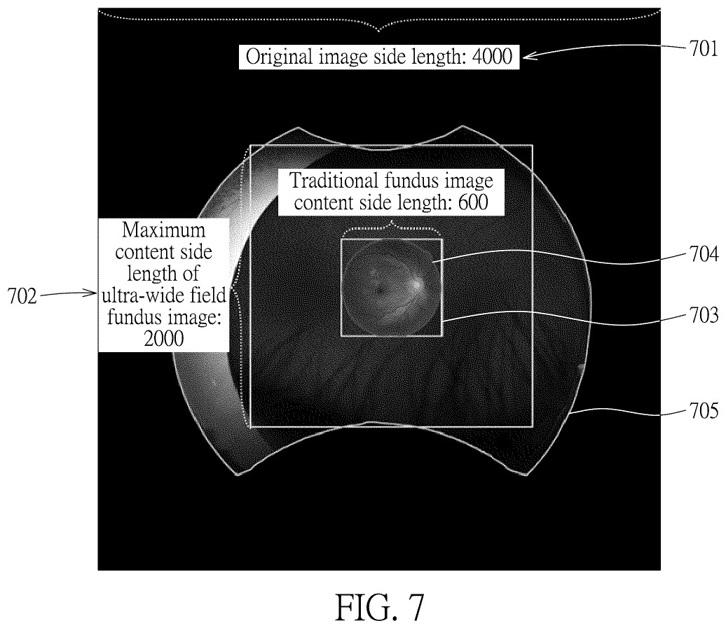

Referring to FIG. 7, the setting regarding side length of the region of interest 602 is related to accessible amount of information content contained in the ultra-wide field fundus image of the patient. For example, the area 701 corresponds to an uncropped ultra-wide field fundus image having original size of 4000×4000 pixels, and an area 703 having size of 600×600 pixels at center of the uncropped ultra-wide field fundus image may contain a same amount of information content as area 704 of a traditional fundus image. Therefore, cropping the ultra-wide field fundus image into a size of 400×400 pixels or 300×300 pixels may appropriately cut down amount of information content in the ultra-wide field fundus image acquired for processing of the neural network model. In another example, considering the ultra-wide field fundus image being a projection of a flattened three-dimensional object on a flat surface, and area 705 corresponding to maximum amount of information content in the ultra-wide field fundus image may vary due to differences between subjects (patients), the ultra-wide field fundus image may also be cropped into a region 702 having size of 2000×2000 pixels. The size of 2000×2000 pixels for cropping may regard as an optimal size to eliminate area with no information outside of area 705 corresponding to maximum amount of information content in a regular ultra-wide field fundus image.

Considering other requirements in diagnosis precision, performance limitations in software apparatus and/or hardware apparatus, and/or optimal efficacy in processing of the neural network model, other convolutional neural network may also be selected for establishing neural network model of the deep learning module 40, and the region of interest 602 for cropping by the cropping module 30 may also set as other sizes (such as but not limited to a size smaller than 4000×4000 pixels) or shapes.

### II. Validating Feasibility of Using Ultra-Wide Field Fundus Image for Detecting and Classifying Type of Cataract

After neural network model of deep learning module 40 is established and region of interesting 602 for cropping by the cropping module 30 is selected, generalization ability of the neural network model of the medical image processing system 100 may be validated using the second dataset having clinical evidence marker data, and feasibility of using ultra-wide field fundus image for detecting and classifying type of cataract may be evaluated.

### Apparatus for Experiment

### I. Ultra-Wide Field Fundus Photography System

The embodiment described herein uses Optos California (P200DTx icg) produced by Optus PLC for shooting ultra-wide field fundus image with an original size of 4000×4000 pixels.

### II. Three-Dimensional Coaxial Lighting Operation Microscope

The embodiment described herein uses ZEISS OPMI Lumera T operation microscope produced by Carl Zeiss Meditec, Inc. for shooting operation coaxial photography video.

### III. Environment for Deep Learning Training

The embodiment described herein establishes the neural network model of the deep learning module 40 under environment with specifications as follows:
1. Central processor: Intel i7-13700KF
2. System memory size: 64 GB
3. Display card: GeForce RTX 3090 24 GB
4. Processing system: Ubuntu 22.04.1
5. Framework of deep learning: Python 3.9 TensorFlow 2.10 (with Keras API)

The apparatus for experiment as described above only shows means for implementing the present disclosure, and may be implemented in other appropriate environments or realized through other software or hardware, where the present disclosure is not limited thereto.

### Model Training

### I. Arrangement of Datasets

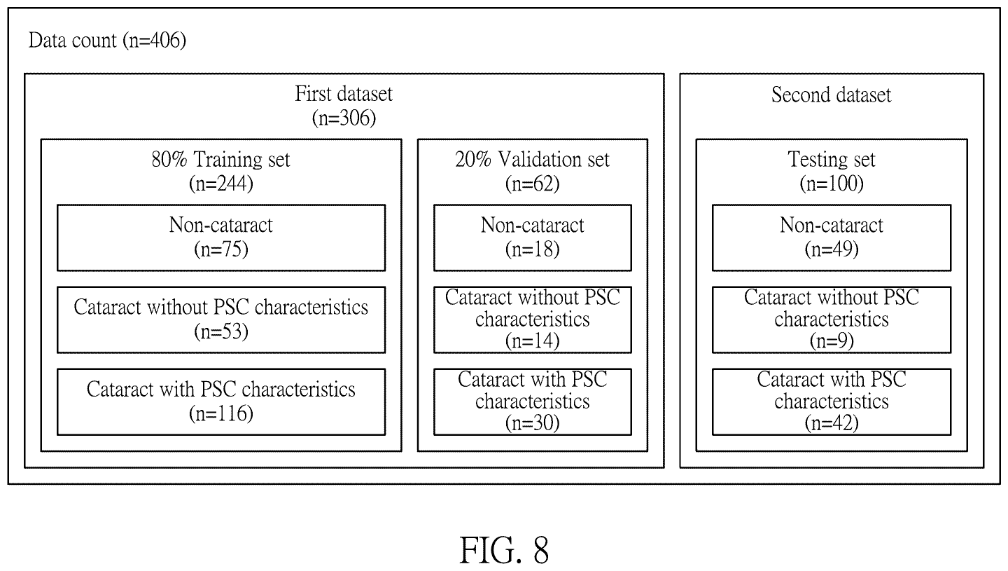

Refer to FIG. 8 and FIG. 5 where arrangement of dataset is described as follows: 80% (244 pieces) of the first dataset (i.e., the 306 pieces of data having only record of ultra-wide field fundus image) may act as training set to train the neural network model to avoid overfitting of the neural network model of the deep learning module 40; 20% (62 pieces) of the first dataset may act as validation set that does not participate in training but to determine efficacy of the neural network model processing unknown data; the second dataset (i.e., the 100 pieces of data having records of clinical evidence marker data) may act as testing set that does not participate in training but to evaluate generalization ability of neural network model and feasibility of using ultra-wide field fundus image for detecting and classifying type of cataract.

### II. Model Structure

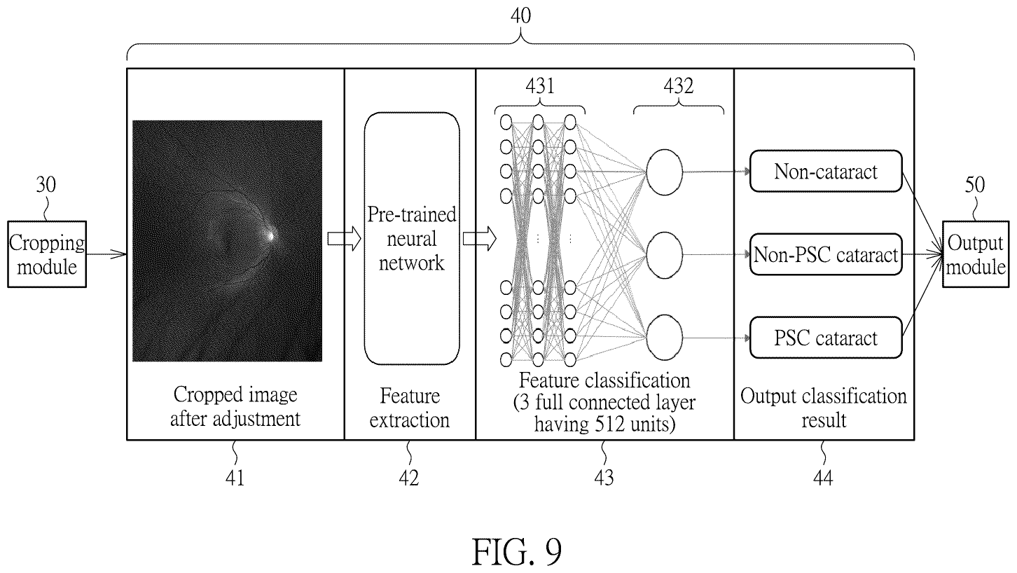

Referring to FIG. 9, the model structure for neural network model of the deep learning module 40 may include an adjustment unit 41, a feature extraction unit 42, a feature classification unit 43 and a result output unit 44. The working mechanism of the deep learning module 40 may include: after the deep learning module 40 acquired cropped image from the cropping module 30, the adjustment unit 41 may implement bilinear interpolation to adjust the cropped image into an input size (e.g., 224×224 pixels) for inputting into the neural network model; the feature extraction unit 42 may realize as a convolutional neural network based on a pre-trained neural network (e.g., the feature extraction unit 42 may be a convolutional neural network containing pre-trained initial weighting of ImageNet, where the pre-trained weightings for base model in transfer learning may be acquired from Keras Applications provided by TensorFlow Keras API) and may be used to extract features from adjusted cropped image; the feature classification unit 43 may realize as a fully connected network 431 of three fully connected layers for analyzing features (e.g., analyzing shadow features in the ultra-wide field fundus image) and a classification layer 432 (output layer) for concluding (outputting) an analysis result (e.g., the analysis result may set as one of the following classifications: cataract with PSC characteristics, cataract without PSC characteristics, and non-cataract) according to classification of features by the fully connected network 431, where each of the fully connected layer may include random initial weightings and 512 flattened neurons, and the classification layer 432 may include 3 flattened neurons; and the result output unit 44 may be used to take the analysis result from the classification layer 432 and output a lens opacification type to the output module 50.

In the structure shown in FIG. 9, the feature extraction unit 42 may utilize the same activation function as the pre-trained neural network (the base model for transfer learning); the fully connected network 431 may utilize ELU activation function and apply complexity of regularization penalty parameter to avoid overfitting; and the classification layer 432 may utilize SoftMax activation function to combine all output from the deep learning module 40 to be 1, so as to compute a percentage of the deep learning module 40 classifying the cropped image into a classification of a lens opacification type (one of the three lens opacification types).

In some alternative embodiments, the structure of neural network model of the deep learning module 40 may be realized in other means, such as integrating the above units of the deep learning module 40 as one unit for determining classification of the lens opacification type. For example, under condition that the pre-trained neural network has sufficient efficacy for processing, the feature classification unit 43 may be omitted and the feature extraction unit 42 may immediately generate an analysis result based on the features upon feature extraction.

### III. Training Parameters

Refer to Table 1 below, the training parameters set for neural network model of the deep learning module 40 may include the following: RMSprop for optimizer; 1×10⁻⁴ for initial learning rate; automatically dropping learning rate to half (at most dropping to 1×10⁻⁵) when offset with validation set during training (validation set loss value) has not drop for a continuous 5 cycles (meaning the neural network model has start to become overfitting to the training); and automatically stopping training when offset with validation set during training (validation set loss value) has risen or not dropped for a continuous 10 cycles.

TABLE 1
training parameters

| Parameter | Value |
|---|---|
| Optimizer | RMSprop |
| Initial Learning | 1 × 10⁻⁴ (0.0001) |
| Rate | |
| Dataset Size | 64 |
| Learning Rate | Dropping to half when validation set loss |
| | value has not drop for a continuous 5 cycles |
| | Minimum value: 1 × 10⁻⁵ (0.00001) |
| Stop Learning | Stop learning when validation set loss value |
| | has not dropped for a continuous 10 cycles |

### IV. Data Augmentation

Table 2 below describes the data augmentation transform variations used by the neural network model of the deep learning module 40. Before training takes place, the ultra-wide field fundus image in each piece of data in the training set may be randomly applied to the data augmentation transform variations of Table 2 to prevent the neural network model from applying learning on the same data multiple times, lowering recognition ability towards new data, and becoming overfitting due to an overly small dataset.

TABLE 2
Data augmentation transform variations

| Transform | Amount of transform |
|---|---|
| Horizontal | −20%~20% |
| translation | |
| Vertical | −20%~20% |
| translation | |
| Scaling | −20%~20% |
| Horizontal | |
| flipping | |

### V. Stratified K-Fold Cross Validation

Different sampling method for arranging training set and validation set may result in different efficacy for the neural network model. Therefore, stratified 5-fold validation is implemented to prevent the neural network model from gaining deviation due to sampling means. For example, the first dataset shown in FIG. 8 may be divided into 5 portions, where the proportion of the classification of lens opacification type (e.g., the proportion of data corresponding to cataract with PSC characteristics, data corresponding to cataract without PSC characteristics, and data corresponding to non-cataract) for data in each portion are the same for those of the first dataset. During each of the 5 modeling training iterations for the neural network model, 1 portion of the 5 portions may act as validation set (i.e., the 20% of the first dataset are being used as validation set and does not participate in training), while the other 4 portions may act as training set (i.e., 80% of the first dataset are being used as training set). An average accuracy may be calculated from the efficacy of the neural network model during the 5 model training iterations.

### Evaluation Indicators

### I. Confusion Matrix

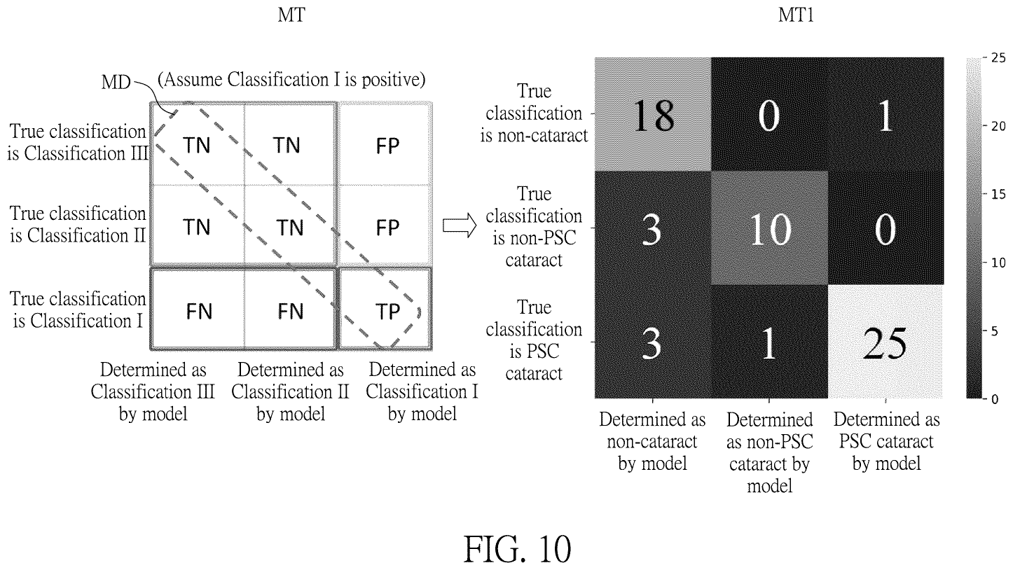

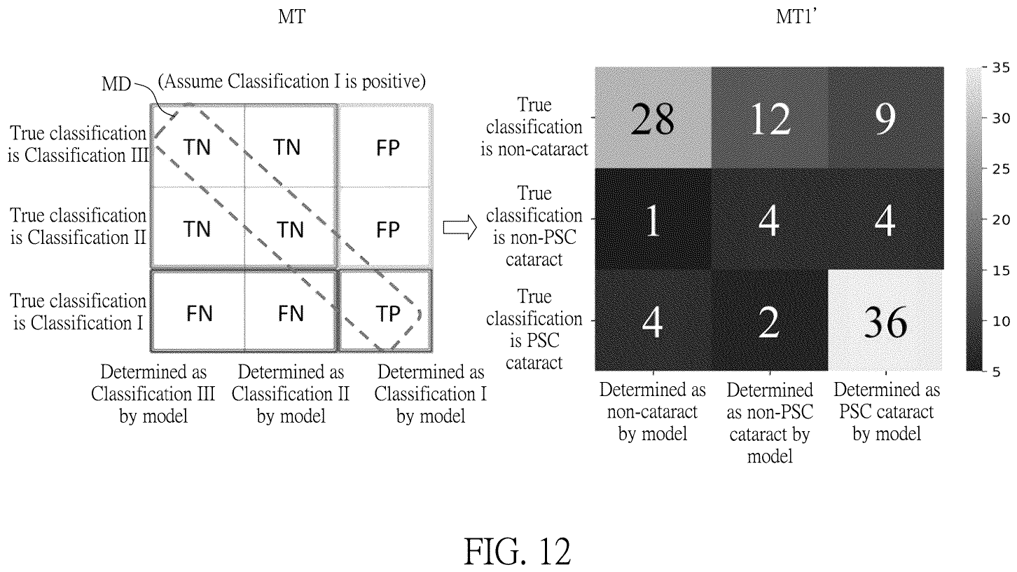

As described in matric MT on the left of FIG. 10 or FIG. 12, confusion matrix is often used as evaluation indicators for multi-classification problems. The confusion matrix may present as a two-dimensional square matrix, where number of columns and rows may correspond to number of classifications targeted for recognition, each column corresponds to a specific classification of a piece of data guessed by the neural network model, and each row corresponds to an actual classification a piece of data should be assigned to. In other words, each element in the matrix MT represents a total number of data with actual classification shown by a corresponding row but being guessed by the neural network model to be a specific classification as shown by a specific column. Further, each element on main diagonal line MD of the matrix MT represents a total number of data in the dataset with actual classification shown by a corresponding row being correctly guessed by the neural network model to be a corresponding classification as shown by a corresponding column.

As shown in the matrix MT, confusion matrix may be used to evaluate true positive (TP), true negative (TN), false positive (FP), and false negative (FN) of the neural network model of the deep learning module 40 in performing detecting and classifying cataract. For example, classification I may represent positive data actually related to targeted cataract in the dataset, TP represents number of positive data being correctly determined as positive, TN represents number of negative data being correctly determined as negative, FP represents number of negative data being mistaken as positive, and FN represents number of positive data being mistaken as negative. Accordingly, the matrix MT may also present corresponding positions of TP data, TN data, FP data and FN data, respectively.

The confusion matrix (matrix MT1) resulted from performance of the trained neural network model of the deep learning module 40 will be discussed below.

### II. Sensitivity, Specificity and Accuracy

The TP data, FP data, TN data and the FN data of confusion matrix as explained above may be used to derive sensitivity, specificity and accuracy of the trained neural network model of the deep learning module 40 in identifying classification of the data in the dataset. The indicators may be explained in detail as follows:

### 1. Sensitivity

Sensitivity may be computed by TP/(TP+FN) and may represent the proportion of positive data being correctly determined as positive. In recognizing lens opacification type related to cataract as described in the present disclosure, the classification for the positives may be set as cataract with PSC characteristics, and sensitivity may represent the proportion of patient having cataract with PSC characteristics being correctly determined to having such condition.

### 2. Specificity

Specificity may be computed by TN/(TN+FP) and may represent the proportion of negative data being correctly determined as negative. In recognizing lens opacification type related to cataract as described in the present disclosure, the classification for the positives may be set as cataract with PSC characteristics, and specificity may represent the proportion of patient that is non-cataract being correctly excluded during diagnosis.

### 3. Accuracy

Accuracy may be computed by (TP+TN)/(TP+FN+FP+TN) and may represent the proportion of data being correctly assigned to a correct classification, and may act as basis for determining classification efficacy of the neural network model as a whole.

### Result of Experiment

### Result of Experiment I

### I. Model Efficacy in Using Different Base Model for Transfer Learning and Using Different Center Cropping Sizes

Table 3 below is efficacy data of the neural network model in detecting and classifying cataract after being established using different sizes for cropping the ultra-wide field fundus image through the cropping module 30, different base models from different pre-trained neural networks for transfer learning through the deep learning module 40, and stratified 5 fold cross validation.

In table 3, setting a center cropping size of 1200×1200 pixels for the cropping module 30 is beneficial in achieving optimal classification efficacy (i.e. the emphasized 80.1%) regardless of type of pre-trained neural network used for establishing the neural network model. On the other hand, selecting ConvNeXt-Tiny as pre-trained neural network for establishing neural network model and setting a center cropping size of 1400×1400 pixels for the cropping module 30 may achieve the highest classification accuracy (i.e., the emphasized italics 81.7%).

TABLE 3
relationship between model average accuracy and center cropping size

| | Center cropping size | | | | | | | | | |
|---|---|---|---|---|---|---|---|---|---|---|
| Model | 300 | 400 | 600 | 800 | 1000 | 1200 | 1400 | 1600 | 1800 | 2000 |
| ResNet50V2 | 79.4% | 80.7% | 77.5% | 76.8% | 79.1% | 81.0% | 78.8% | 77.1% | 75.8% | 75.8% |
| InceptionV3 | 79.1% | 79.1% | 80.7% | 79.4% | 79.1% | 79.8% | 78.1% | 77.4% | 78.1% | 75.5% |
| Xception | 77.5% | 78.8% | 77.8% | 78.8% | 81.0% | 81.0% | 78.4% | 79.7% | 77.1% | 78.1% |
| EfficientNetV2-S | 78.8% | 77.8% | 77.5% | 79.1% | 80.1% | 78.4% | 79.7% | 79.7% | 79.4% | 75.8% |
| ConvNext-Tiny | 79.4% | 78.8% | 77.4% | 80.7% | 79.1% | 80.4% | 81.7% | 77.1% | 75.8% | 77.5% |
| Average | 78.8% | 79.8% | 78.2% | 79.0% | 79.7% | 80.1% | 79.4% | 78.2% | 77.2% | 76.5% |

Based on the above, using a center cropping size of 1200×1200 to crop the ultra-wide field fundus images for detecting and classifying cataract may enable the neural network model to (a) avoid receiving unnecessary information in comparison to a larger center cropping size or (b) avoid losing shadow features of the cataract at outer edge of the retina in comparison to a smaller center cropping size.

However, also observed is that the neural network model established using ConvNeXt-Tiny may achieve the best classification accuracy using center cropping size of 1400×1400 pixels, and the neural network model established using other models may also achieve higher classification accuracy (e.g., higher than 80%) using different center cropping sizes respectively. Therefore, the cropping size set for the cropping module 30 cropping the ultra-wide field fundus image and the pre-trained neural network for establishing the neural network model of the deep learning module 40 may be configured based on operational, software or hardware requirements of the medical image processing system 100 (e.g., selecting a model with faster training speed as pre-trained neural network for establishing the neural network model), or even using elements other than the ones listed in table 3.

Model efficacy of the present disclosure proven by validation set and testing set (as shown in FIG. 8) may be described below under the settings of: the cropping module 30 may generate cropped image with size of 1400×1400 pixels, deep learning module 40 includes a neural network model established using ConvNeXt-Tiny, and the medical image processing system 100 is configured to detect and classify cataract with PSC characteristics.

### II. Proving Model Efficacy Using the Validation Set

The matrix MT1 to the right of FIG. 10 and table 4 below shows TP data, TN data, FP data and FN data of the deep learning module 40 using the validation set for detecting and classifying cataract and sensitivity, specificity, and accuracy of the deep learning module 40 using the validation set for determining cataract with PSC characteristics, where each classification and data in the matrix MT1 represents a same meaning as those labeled in matrix MT1 to the left of FIG. 10.

TABLE 4
Efficacy of model using validation set

| Lens opacification type | Sensitivity | Specificity | Accuracy |
|---|---|---|---|
| Non-cataract | 94.7% | 85.7% | 88.5% |
| Cataract without PSC | 76.9% | 97.9% | 93.4% |
| characteristics | | | |
| Cataract with PSC | 86.2% | 96.8% | 91.8% |
| characteristics | | | |

Therefore, it is proven that the neural network model of the deep learning module 40 may still maintain a desired efficacy while using validation set, which does not take part in training, for detecting and classifying cataract and achieve sensitivity, specificity, and accuracy at over 85%. Therefore, neural network model without overfitting is capable of using dataset labeled by clinicians to learn detecting and determining type of cataract using ultra-wide field fundus image.

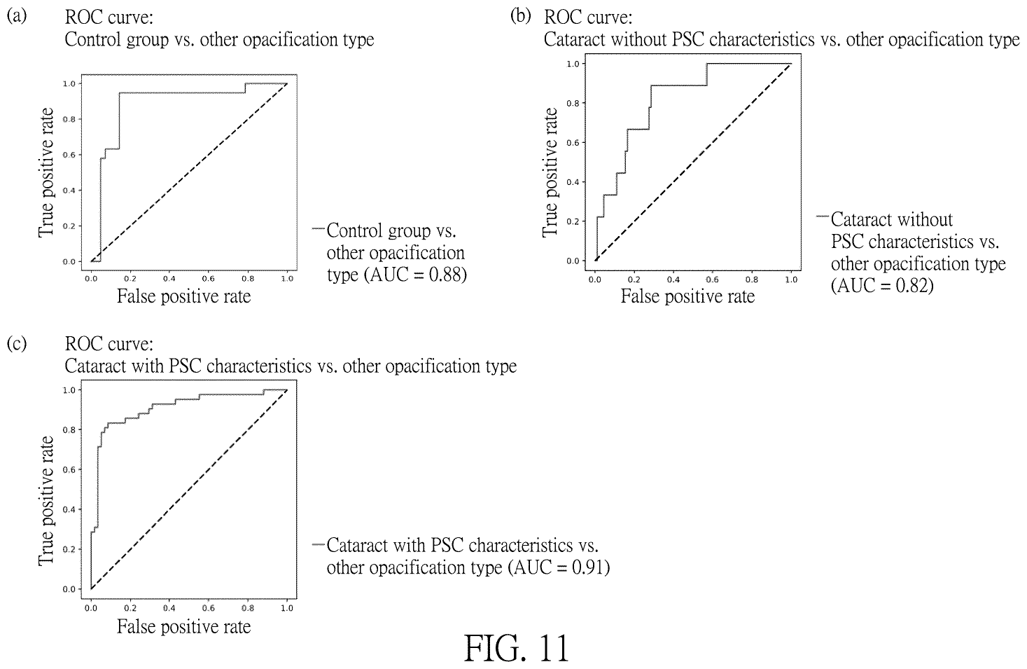

FIG. 11 shows receiver operating characteristics (ROC) curves and areas under the curve (AUC) regarding comparisons of the neural network model of the deep learning module 40 predicting the three lens opacification type using the validation set. As shown, the neural network model in the present disclosure may achieve AUC far bigger than 0.5 regardless in identifying lens opacification type corresponding to (a) non-cataract, (b) cataract without PSC characteristics or (c) cataract with PSC characteristics based on a given ultra-wide field fundus image. Therefore, the neural network model of the present disclosure is capable of randomly guessing lens opacification type with fine classification capability.

### Result of Experiment II

### I. Proving Model Efficacy Using the Testing Set

The matrix MT1′ to the right of FIG. 12 and table 5 below shows TP data, TN data, FP data and FN data of the deep learning module 40 using the testing set for detecting and classifying cataract and sensitivity, specificity, and accuracy of the deep learning module 40 using the testing set for determining cataract with PSC characteristics, where each classification and data in the matrix MT1′ represents a same meaning as those labeled in matrix MT1 to the left of FIG. 12.

TABLE 5
Efficacy of model using testing set

| Lens opacification type | Sensitivity | Specificity | Accuracy |
|---|---|---|---|
| Non-cataract | 57.1% | 90.2% | 74% |
| Cataract without PSC | 44.4% | 84.6% | 81% |
| characteristics | | | |
| Cataract with PSC | 83.7% | 77.6% | 81% |
| characteristics | | | |

Therefore, it is proven that the neural network model of the deep learning module 40 may achieve fine sensitivity (83.7%), specificity (77.6%), and accuracy (81%) in determining cataract with PSC characteristics, meaning using ultra-wide field fundus image in detecting and determining cataract with PSC characteristics by the neural network model is feasible in clinical practice. Further, the neural network model also achieves 90.2% in specificity in identifying non-cataract (control group), meaning the neural network model is capable of partitioning data of patients without mistaken a patient as a healthy individual (non-cataract). Moreover, the neural network model may also achieve 81% in accuracy in identifying cataract without PSC characteristics.

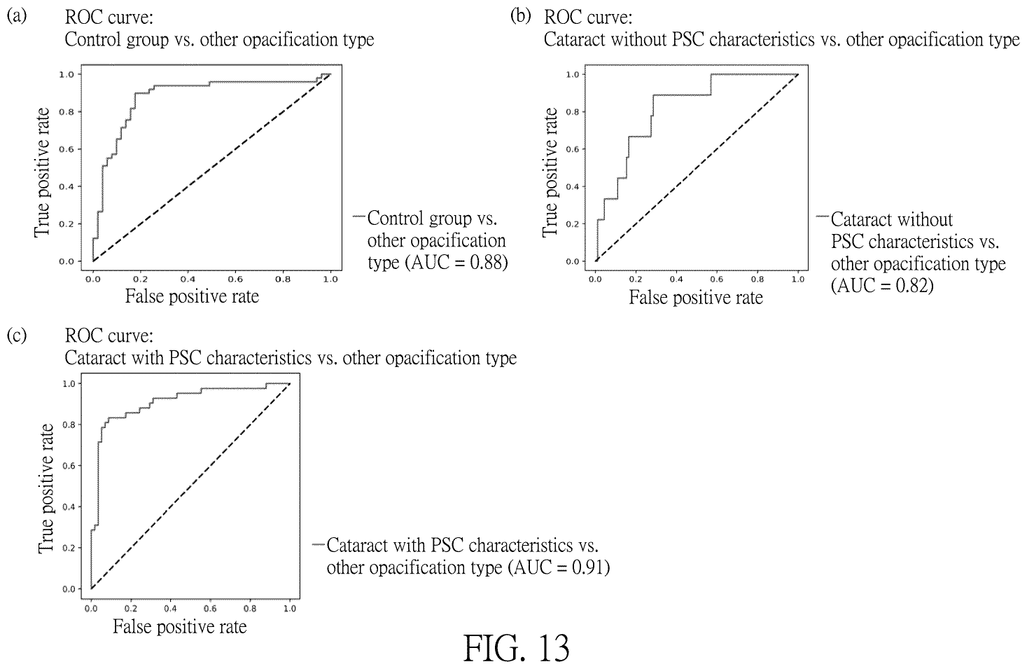

FIG. 13 shows ROC curves and AUC regarding comparisons of the neural network model of the deep learning module 40 predicting the three lens opacification type using the testing set. As shown, the neural network model in the present disclosure may achieve AUC far bigger than 0.5 regardless in identifying lens opacification type corresponding to (a) non-cataract, (b) cataract without PSC characteristics or (c) cataract with PSC characteristics based on a given ultra-wide field fundus image. Therefore, the neural network model of the present disclosure is capable of randomly guessing lens opacification type with fine accuracy. It is especially fund that the neural network model may achieve AUC of 0.91 in detecting and identifying cataract with PSC characteristics.

### Other Results in the Experiments

The results of the medical image processing system 100 detecting and classifying cataract for treating posterior polar cataract (PPC) will be described below.

### Result of Experiment I′

For example, a dataset having 549 pieces of data with records of ultra-wide field fundus image gathered from a period of 2 years may be used to establish neural network model of the deep learning module 40. In here, the dataset may be partitioned into first dataset (446 pieces) and second dataset (103 pieces) in a same manner as described in FIG. 8, where the first dataset (including the training set and the validation set) may include 152 pieces of data being labeled as non-cataract (control group), 96 pieces of data being labeled as cataract without PPC characteristics and 198 pieces of data being labeled as cataract with PPC characteristics, and the second dataset (testing set) may include 52 pieces of data being labeled as non-cataract (control group), 9 pieces of data being labeled as cataract without PPC characteristics and 42 pieces of data being labeled as cataract with PPC characteristics.

### I. Model Efficacy in Using Different Base Model for Transfer Learning and Using Different Center Cropping Sizes

Referring to table 6 below, among all combinations of sizes for cropping the ultra-wide field fundus image and different base models from different pre-trained neural networks for transfer learning, a combination of center cropping size of 1400×1400 pixels and a neural network model established based on pre-trained neural network ConvNeXt-Tiny, the dataset described above and stratified 5 fold cross validation is selected for having the best efficacy (with accuracy of 82% marked in emphasized italic) and being used to demonstrate model efficacy.

TABLE 6
relationship between model average accuracy
and center cropping size of the best model

| | Center cropping size | | | | | | | | | |
|---|---|---|---|---|---|---|---|---|---|---|
| model | 300 | 400 | 600 | 800 | 1000 | 1200 | 1400 | 1600 | 1800 | 2000 |
| ConvNeXt- | 76% | 77% | 79% | 81% | 80% | 80% | 82% | 76% | 79% | 79% |
| Tiny | | | | | | | | | | |

### II. Proving Model Efficacy Using the Validation Set

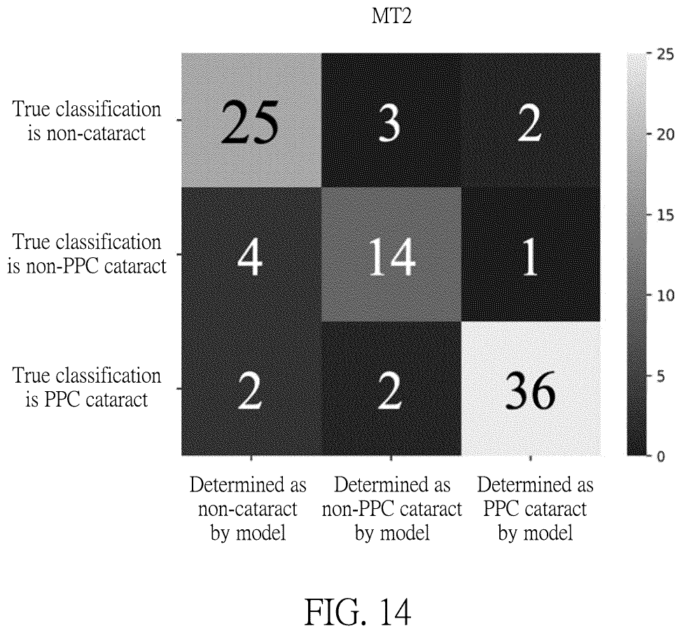

As shown in FIG. 14, matrix MT2 describes TP data, TN data, FP data and FN data of the deep learning module 40 using the validation set for detecting and classifying cataract. From here, it can be seen that medical image processing system 100 may achieve accuracy of 84% at detecting and identifying cataract with PPC characteristics even with using validation set that does not take part in training.

### Result of Experiment II′

### I. Proving Model Efficacy Using the Testing Set

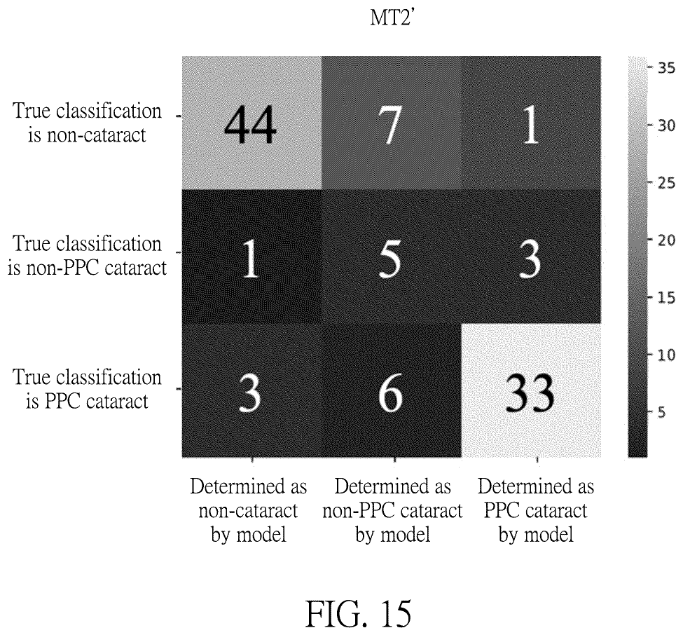

As shown in FIG. 15, matrix MT2′ describes TP data, TN data, FP data and FN data of the deep learning module 40 using the testing set for detecting and classifying cataract. With evidence of clinical evidence marker data of the second dataset, it can be seen that the medical image processing system 100 may achieve overall accuracy of 80% at detecting and identifying cataract with PPC characteristics in clinical practice. Further looking at the TN data and TP data of the medical image processing system 100 in identifying non-cataract, cataract without PPC characteristics and cataract with PPC characteristics, the accuracy may reach up to 88%, 84% and 87%.

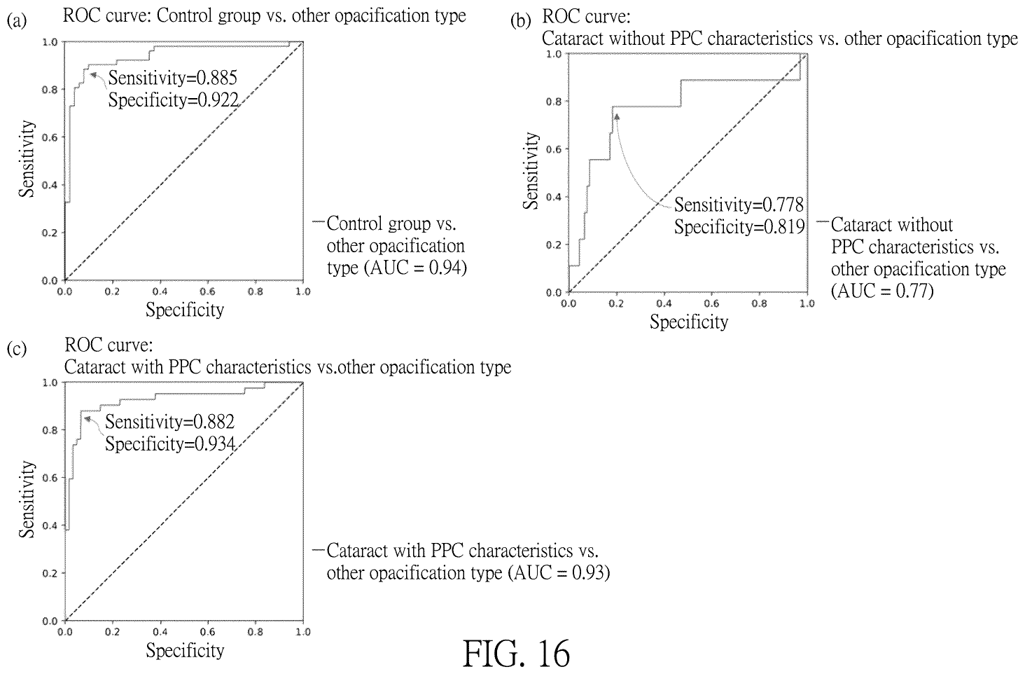

FIG. 16 shows ROC curves and AUC regarding comparisons of the neural network model of the deep learning module 40 predicting the three lens opacification type using the testing set. As shown, the neural network model in the present disclosure may achieve high AUC regardless in identifying lens opacification type corresponding to (a) non-cataract, (b) cataract without PSC characteristics (which has the lowest AUC of 0.77) or (c) cataract with PSC characteristics based on a given ultra-wide field fundus image. It is especially fund that the neural network model may achieve specificity of 93.4% in identifying cataract with PPC characteristics, meaning the neural network model of the present disclosure is highly capable of detecting and identifying cataract with PPC characteristics.

### CONCLUSION

The medical image processing system 100 of the present disclosure may be used to learn ability of clinicians to identify a targeted type of cataract via ultra-wide field fundus image. The neural network model after training is also proven by clinical evidence marker data to be feasible and highly capable of detecting and classifying cataract using the ultra-wide field fundus image. The medical image processing system 100 may also efficiently crop the ultra-wide field fundus image to eliminate non-fundus information without losing necessary features for detecting and classifying cataract. In the example of setting center cropping size for the ultra-wide field fundus to be 1400×1400 pixels and ConvNeXt-Tiny to be base model for transfer learning in establishing the neural network model, not only the medical image processing system 100 may achieve sufficient accuracy in classifying cataract even with validation set that does not take part in training, but also the training of the neural network model using the training set is prevented from leading to overfitting. Therefore, the medical image processing system 100 of the present disclosure may act as an initial filtering tool for assisting clinicians in diagnosing cataract, cutting down professional human resource in clinical diagnosing, reminding risk of related complications of the patient to the clinician before cataract surgery is performed and enable clinicians to be more careful and prevent complications during surgery. The medical image processing system 100 may also integrate into edge computing device and/or existing apparatus in the eye clinic to achieve telemedicine. For example, a patient may be required to have ultra-wide field fundus image photographed, and the clinicians may remotely access the corresponding data for detecting and classifying cataract.

Based on the above, medical image processing system, method and computer readable medium thereof may be realized through accessibility of ultra-wide field fundus image, and is beneficial for realizing telemedicine due to patients not being required for dilating pupils, applicable for automatic screening for cataract, little to no disturbance on regular living and working conditions of the patient, and applicable for remote areas with insufficient medical resources. Further, application of deep learning may greatly increase detection rate of cataract, decrease false negatives, decrease diagnosis time wasted at eye clinics, and increase diagnosis efficiency. Further, application of ultra-wide field fundus image may enable visualized communication and improve communication efficiency between clinicians and patients.

Those skilled in the art will readily observe that numerous modifications and alterations of the device and method may be made while retaining the teachings of the disclosure. Accordingly, the above disclosure should be construed as limited only by the metes and bounds of the appended claims.

What is claimed is:

1. A medical image processing system, comprising:
a data acquisition module configured to acquire an ultra-wide field fundus image;
a cropping module coupled with the data acquisition module and configured to crop the ultra-wide field fundus image into a cropped image; and
a deep learning module coupled with the cropping module and configured to detect and determine classification of a lens opacification type corresponding to the cropped image.

2. The medical image processing system of claim 1, wherein:
the lens opacification type corresponds to a type of cataract; and
the cropped image comprises a region of interest of the ultra-wide field fundus image.

3. The medical image processing system of claim 1, wherein the deep learning module comprises:
a feature extraction unit based on a pre-trained neural network and configured to extract a feature of the cropped image; and
a result output unit configured to output the classification of the lens opacification type according to analysis result of the feature.

4. The medical image processing system of claim 1, wherein:
the classification of the lens opacification type is detected and classified through a shadow feature of the ultra-wide field fundus image by the deep learning module; and
the shadow feature is a projected feature of cataract on a retina.

5. A medical image processing method, comprising:
a data acquisition module acquiring an ultra-wide field fundus image;
a cropping module cropping the ultra-wide field fundus image into a cropped image; and
a deep learning module detecting and determining classification of a lens opacification type corresponding to the cropped image.

6. The medical image processing method of claim 5, wherein:
the lens opacification type corresponds to a type of cataract; and
the cropped image comprises a region of interest of the ultra-wide field fundus image.

7. The medical image processing method of claim 5, wherein the deep learning module detecting and determining the classification of the lens opacification type corresponding to the cropped image comprises:
a feature extraction unit extracting feature of the cropped image; and
a result output unit outputting the classification of the lens opacification type according to analysis result of the feature.

8. The medical image processing method of claim 7, wherein:
the feature extraction unit is based on a pre-trained neural network;
the feature is shadow feature of the ultra-wide field fundus image; and
the shadow feature is a projected feature of cataract on a retina.

9. A computer readable medium storing computer executable instruction, wherein the computer executable instruction is executed to perform the medical image processing method of claim 5.
